(19) United States
(12) Patent Application Publication
(10) Pub. No.: US 2010/0234731 A1
(43) Pub. Date: Sep. 16, 2010

(54) Automatic Ultrasonic Doppler Measurements

(75) Inventors: Haiyuan Lu, Bothell, WA (US); Dan Skyba, Snohomish, WA (US)

Correspondence Address: PHILIPS INTELLECTUAL PROPERTY & STANDARDS, P.O. BOX 3001, Briarcliff Manor, NY, 10510-8001, US

(73) Assignee: KONINKLIJKE PHILIPS ELECTRONICS, N.V., EINDHOVEN (NL)

(21) Appl. No.: 12/161,379

(22) PCT Filed: Jan. 22, 2007
(86) PCT No.: PCT/IB07/50216

§ 371 Date: Jul. 18, 2008

Related U.S. Application Data
(60) Provisional application No. 60/762,628, filed on Jan. 27, 2006.

(51) Int. Cl. A61B 8/06

(52) U.S. Cl. 600/454

(57) ABSTRACT

An ultrasonic diagnostic imaging system produces a spectral Doppler display on which automated measurements may be made. The waveform is analyzed by the ultrasound system to identify the peak velocity of each cardiac cycle of the sequence, and the cardiac cycle with the highest peak velocity value. When a measurement tool is launched, the system displays the highest peak velocity cycle and makes the selected measurement on the data of that heart cycle. The system may advantageously use a peak velocity tracing algorithm in support of this feature. The technique can be used with a variety of measurement tools.

This invention relates to medical diagnostic ultrasound systems and, in particular, to ultrasound systems which perform measurements of a Doppler waveform automatically.

In a vascular study numerous blood flow characteristics of a patient are measured and quantified. The clinician begins the exam by acquiring spectral Doppler data from the heart or a blood vessel such as the carotid artery. The patient's vascular anatomy is displayed in a two or three dimensional image on the ultrasound system display and a sample volume cursor is moved to a point in the heart or blood vessel where measurements are to be made. Spectral Doppler data is acquired over time from the sample volume location and displayed as a spectral waveform. Once a steady spectral display is being produced, the clinician begins to record the continuous spectral waveform. After several minutes of the Doppler waveform have been acquired and stored the examination of the patient ends and the clinician reviews, analyzes, and makes measurements of the acquired spectral waveform.

The clinician analyzes the waveform stored by the Cineloop® memory of the ultrasound system by scanning through the spectral data with the trackball on the user interface, looking for a heart cycle of data from which measurements are to be initially made. In order to make measurements of that heart cycle, a measurement program is launched, which can be done either before or after the heart cycle has been located. The clinician may have to mark a cursor on the selected heart cycle at key diagnostic points such as end diastole or at the peak velocity of the waveform in order to key the measurement program to specific points in the data which are to be used in the measurement. The measurement program will then calculate the selected measurement and display a result. This procedure is then repeated for numerous measurements and heart cycles. There can be upwards of 100 such measurements made in a typical vascular or cardiac examination, and this process of launching a measurement program and establishing an initial position for the measurement must be repeated each time. The repetitive nature of these tasks adds a significant amount of time to the overall exam and can lead to repetitive stress injuries to the clinician. Accordingly it is desirable to automate this process so that these measurements can be made more quickly and accurately while reducing repetitive hand motions for the clinician.

In accordance with the principles of the present invention, a diagnostic ultrasound system and method are described which enables a user to automatically compute measurements of a Doppler waveform. The peak velocity values in the waveform are automatically identified by, for example, a peak velocity tracing algorithm, which may be done on the displayed waveform or in the background. The cardiac cycle with the highest peak velocity is identified together with key points of that cardiac cycle waveform. The automatically selected cardiac cycle can be accepted by the clinician or another starting point for measurements can be selected either manually or by another automated heart cycle identification. The accepted cardiac cycle and the values at the key points are then used to make the desired measurements automatically and the results are displayed. The process can be extended to automatically making measurements on heart cycle data preceding or following the peak velocity heartbeat, and/or to making measurements of other high peak velocity cardiac cycles. Among the measurements which can be automated in this way are acceleration/deceleration time, peak systole velocity, minimum diastole velocity, end diastole velocity, time average peak velocity, resistive index, pulsatility index, systolic and diastolic ratio, pressure gradient, velocity time integral, heart rate, slope and time associated with a heart cycle.

In the drawings:

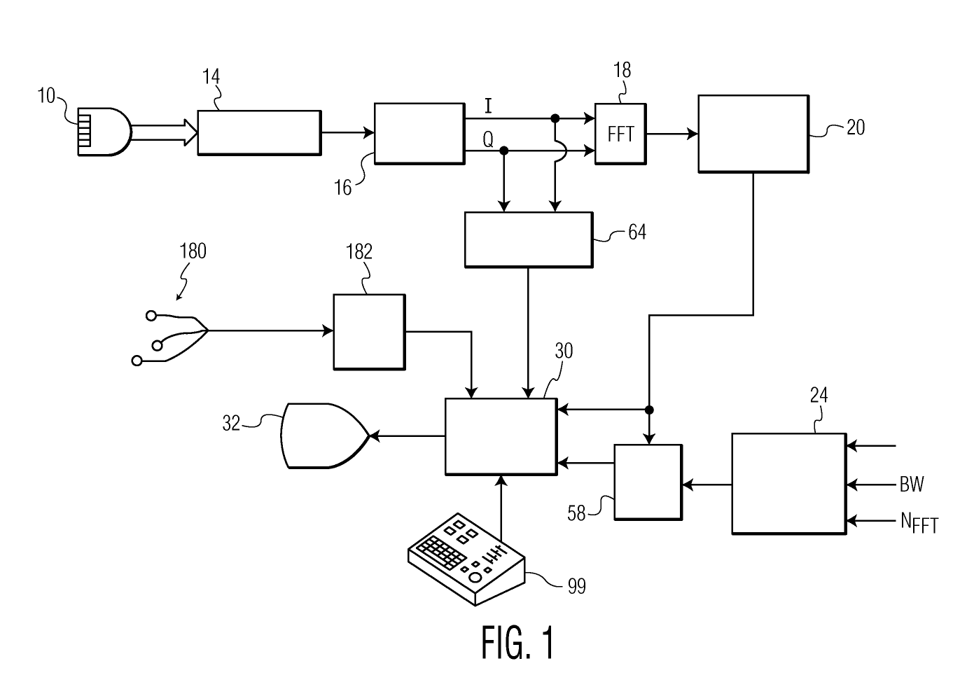

FIG. 1 illustrates in block diagram form an ultrasonic diagnostic imaging system constructed in accordance with the principles of the present invention.

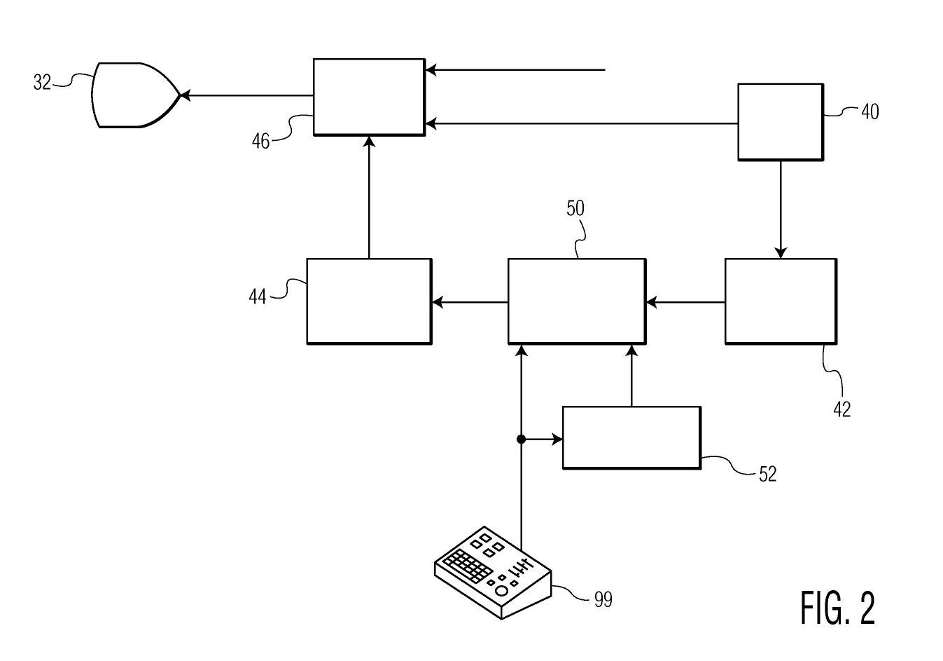

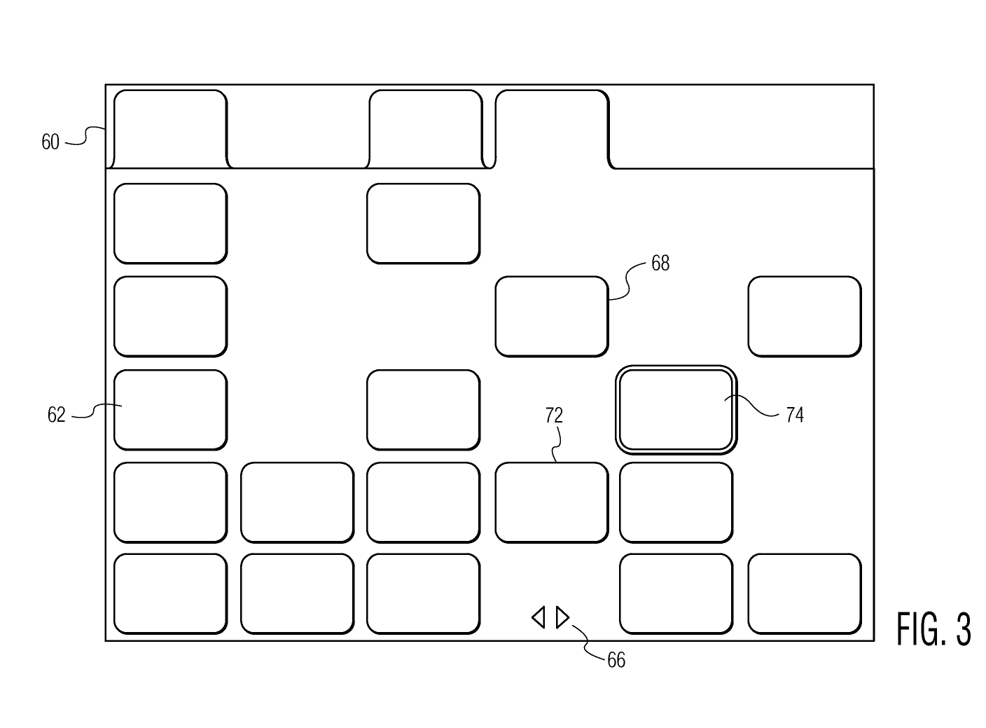

FIG. 2 illustrates in block diagram form a detailed description of the Doppler measurement processor of FIG. 1.

FIG. 3 illustrates a touchscreen control panel of a constructed implementation of the present invention.

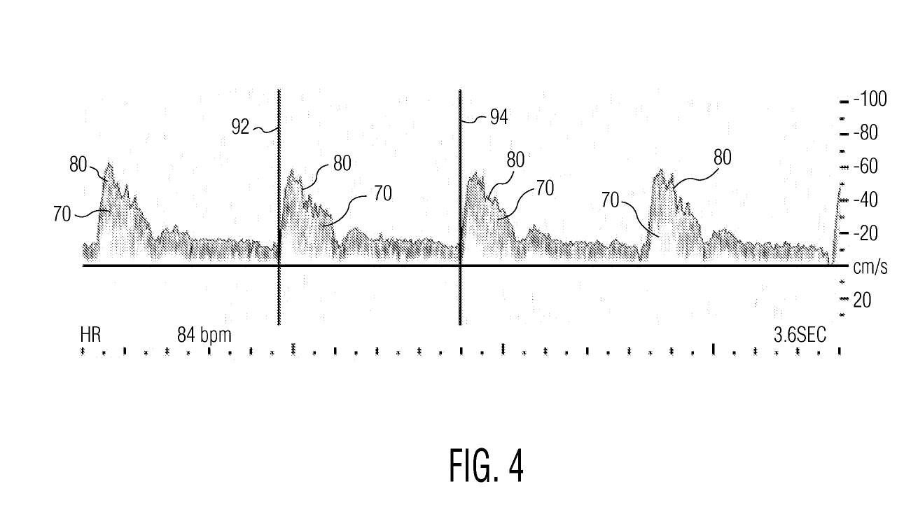

FIG. 4 illustrates a Doppler display in which a heart cycle has been identified in accordance with the principles of the present invention.

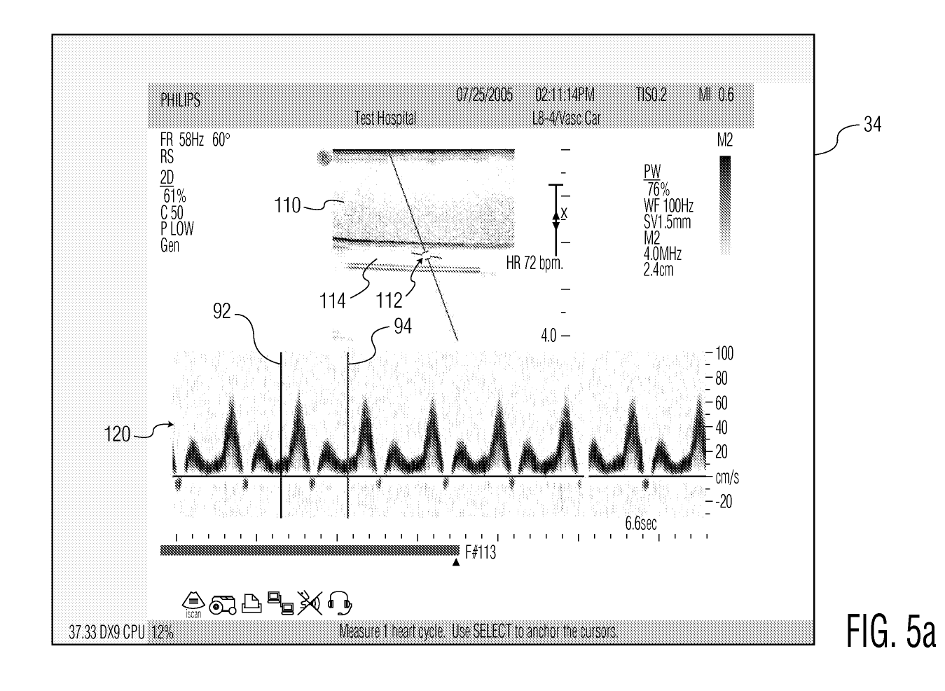

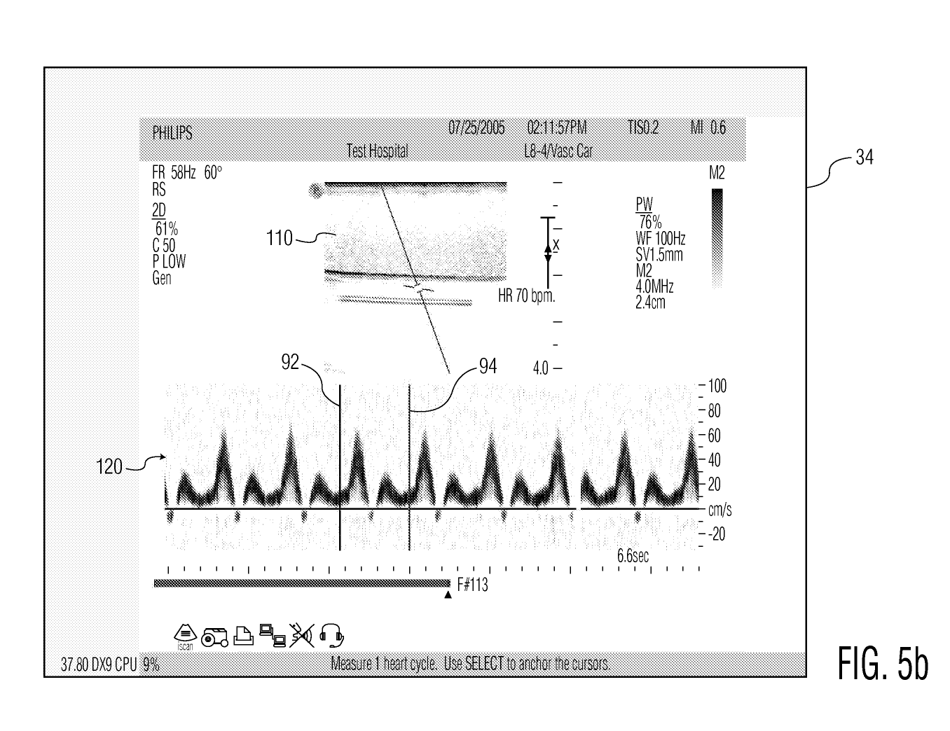

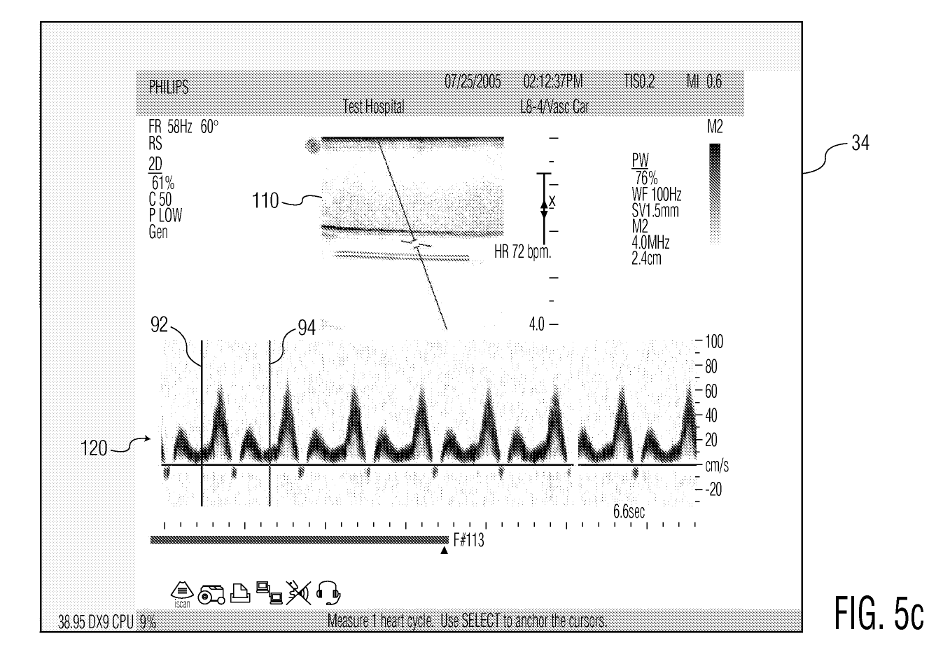

FIGS. 5a, 5b, and 5c illustrate display screens for measuring the heart rate in a Doppler display.

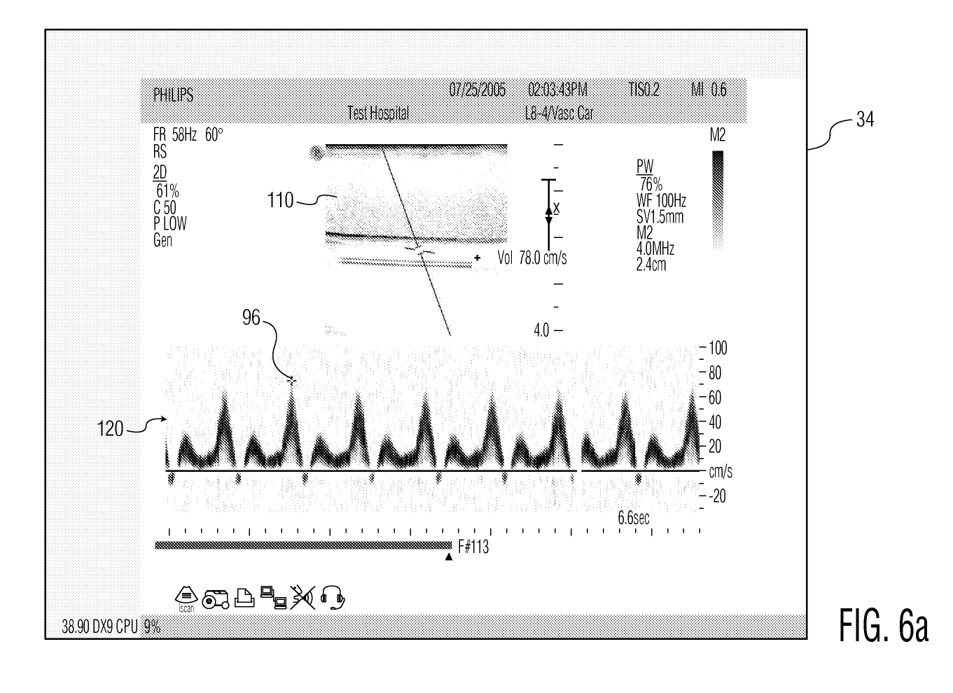

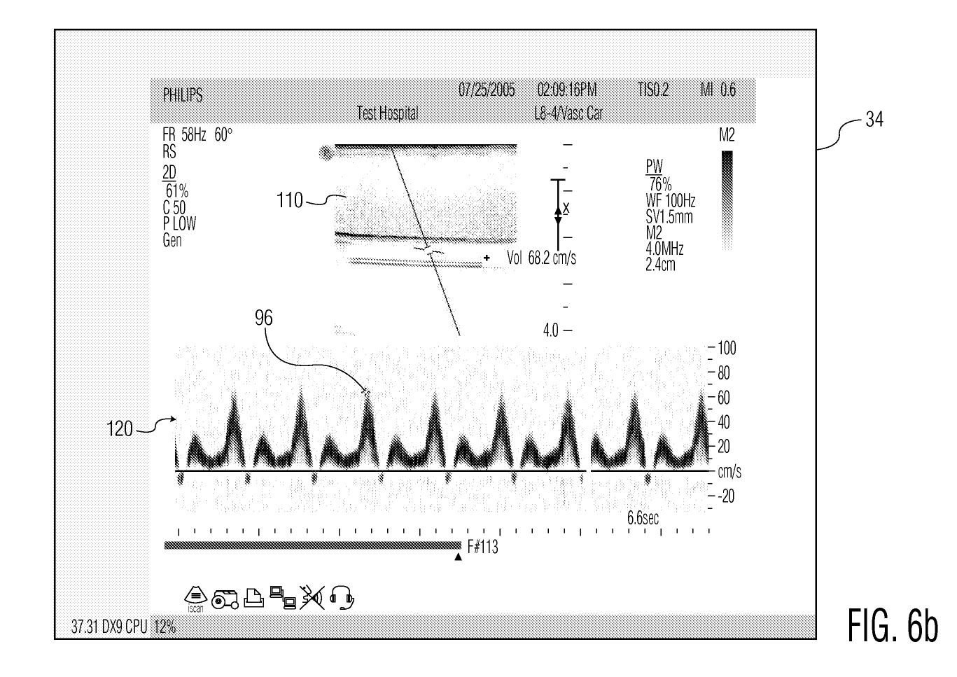

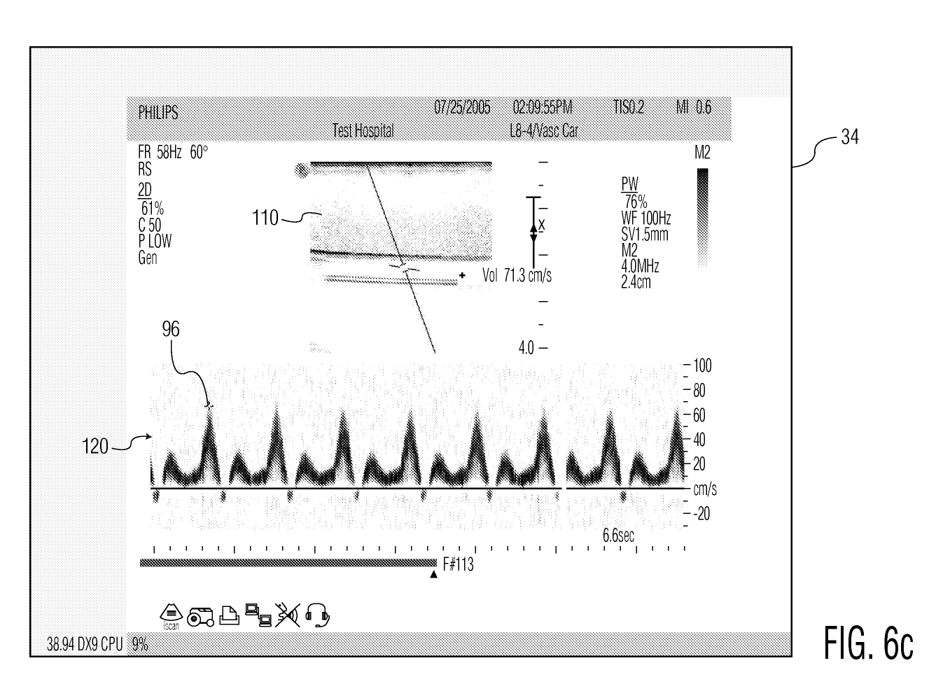

FIGS. 6a, 6b, and 6c illustrate display screens in which the peak velocity value in a Doppler display has been identified in accordance with the principles of the present invention.

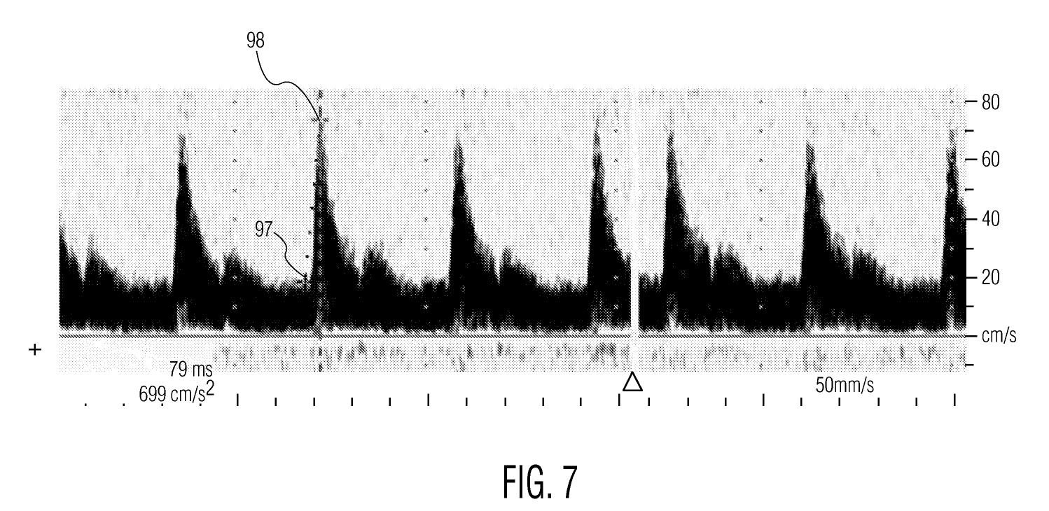

FIG. 7 illustrates the measurement of acceleration time using a time slope tool in accordance with the present invention.

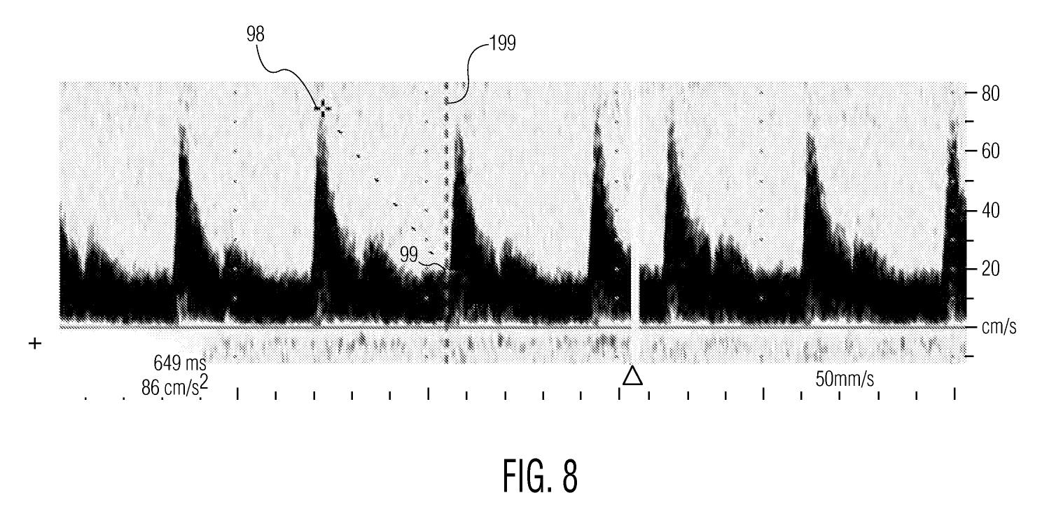

FIG. 8 illustrates the measurement of deceleration time using a time slope tool in accordance with the present invention.

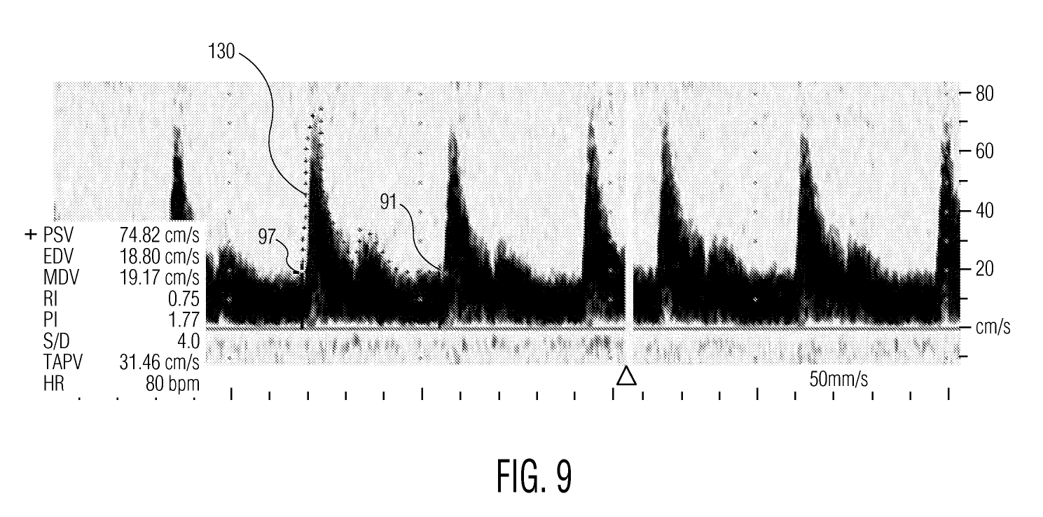

FIG. 9 illustrates the tracing of the Doppler waveform of a cardiac cycle in accordance with the present invention.

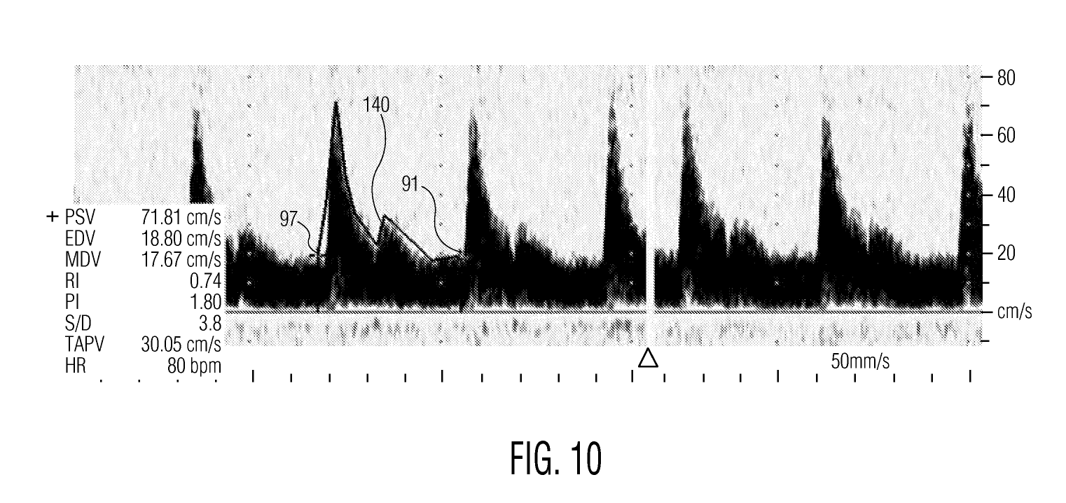

FIG. 10 illustrates the point to point tracing of the Doppler waveform of a cardiac cycle in accordance with the present invention.

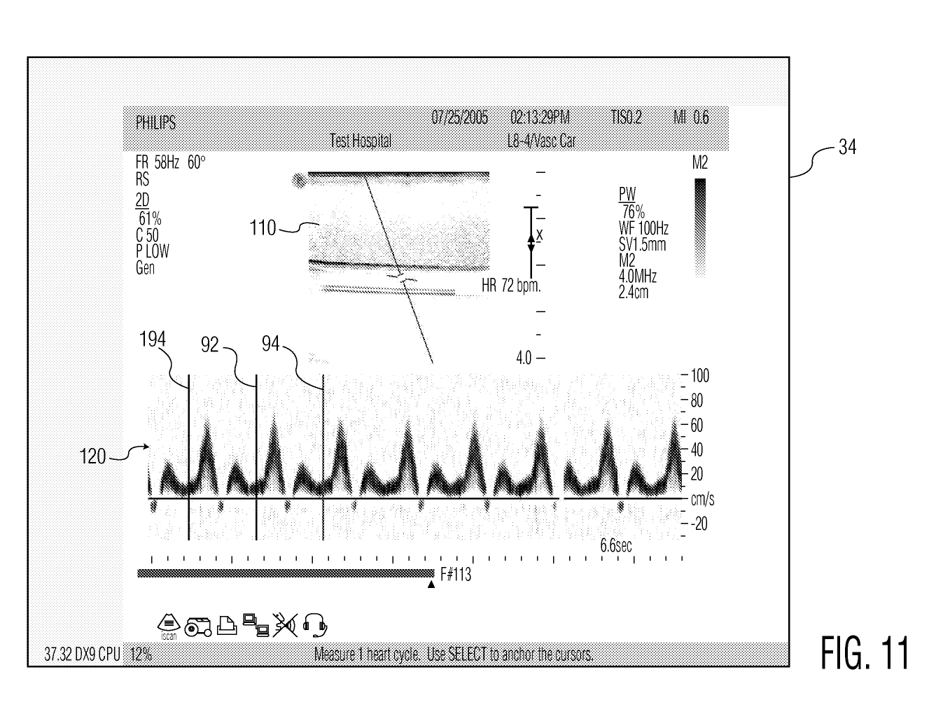

FIG. 11 illustrates the measurement of the heart rate using a 2-cycle average.

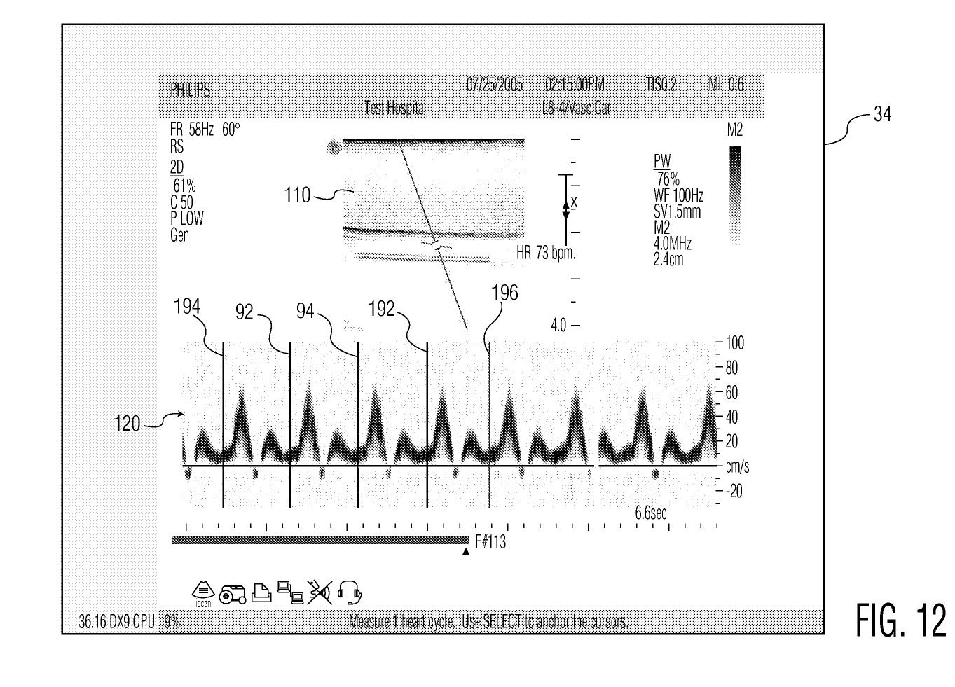

FIG. 12 illustrates the measurement of the heart rate using a 4-cycle average.

Referring first to FIG. 1, an ultrasound system constructed in accordance with the principles of the present invention is shown in block diagram form. Ultrasonic signals are transmitted by a transducer array 10 of an ultrasound probe and the resultant echoes are received by the elements of the transducer array. The received echo signals are formed into a single signal or beam by a beamformer 14. The echo signal information is detected by a Doppler detector 16 which produces quadrature I and Q signal components. A number of such signal components from the site in the body being diagnosed are applied to a Doppler processor 18, one form of which is a fast Fourier transform (FFT) processor, which computes the Doppler frequency shift of the received signals. This basic Doppler data is post-processed by a Doppler post processor 20, which further refines the data by techniques such as wall filtering, gain control, and amplitude compression.

Intermittently during the reception of Doppler echoes, B mode echoes are received. These echoes are also formed into I and Q components which may then be amplitude detected by taking the square root of the sum of the squares of the I and Q values in a B mode image processor 64. The B mode image processor also arranges the B mode echoes into a desired display form by scan conversion. The resultant two or three dimensional image of the anatomy is coupled to a Doppler measurement processor 30 where it is prepared for display with spectral Doppler data and measurement data processed as discussed below.

The post processed Doppler data is applied to a peak velocity detector 58 and the Doppler measurement processor 30. The Doppler measurement processor further processed the Doppler data for the display of a real time sequence of spectral line information. The peak velocity detector compares the Doppler data against a noise threshold NOISEₜₕ to determine the peak velocity point of a spectral line, as discussed more fully in U.S. Pat. Nos. 5,287,753 and 5,634,465. The peak velocity detector 22 may also perform filtering of the Doppler data and may also be used to identify mean velocity levels as discussed more fully in the '753 patent. The Doppler measurement processor 30 thus provides both an anatomical B mode image and a spectral Doppler display with peak and/or mean velocity values automatically identified as the discussed in the aforementioned patents.

The ultrasound display 32 will also preferably show an ECG trace drawn in response to reception of an R-wave signal. The R-wave is the electrical physiological signal produced to stimulate the heart's contraction, and is conventionally detected by an electrocardiograph (ECG). FIG. 1 shows a set of ECG electrodes 180 which may be affixed to the chest of a patient to detect the R-wave signal. The signal is detected and processed by an ECG signal processor 182 and applied to the Doppler measurement processor 30, which displays the ECG waveform in synchronism with the scrolling spectral Doppler display and the anatomical B mode image. The B mode image can be used to locate and display the point in the patient's anatomy at which the spectral information is acquired as illustrated below.

Operation of the Doppler measurement processor 30 in accordance with the principles of the present invention is illustrated by the block diagram of FIG. 2. A spectral Doppler image sequence is stored in a Cineloop memory 40. The spectral Doppler image data is coupled to a display processor 46 for display in synchronism with B mode images from the B mode image processor 64. The spectral Doppler data is also coupled to a waveform peak tracer 42 which may be constructed as described in the aforementioned U.S. Pat. Nos. 5,287,753 and 5,634,465 to detect the peak velocity of each spectral line of the spectral display. By connecting these peak velocity points of the spectral lines the peak velocities of the spectral Doppler display is traced. In accordance with the present invention the waveform peak tracer 42 also identifies and records the peak velocity of each cardiac cycle in the spectral Doppler data being analyzed. This peak normally occurs during each systolic phase of the heart cycle. An individual heart cycle may be identified from inflections in the peak velocity trace or from the ECG signal. In one example of the present invention a heart cycle is identified as the interval between consecutive end diastole points of the spectral display. At the end of this processing the waveform peak tracer 42 will have identified the peak velocity point of all of the heart cycles of the spectral Doppler data being analyzed. This information is coupled to a measurement processor 50.

The measurement processor 50, in addition to receiving velocity peak information from the waveform peak tracer, receives control signals from the user interface 99 and measurement tools from a measurement tool store 52. A “measurement tool” is a software program which analyzes ultrasound data an performs a specific measurement using the data. Examples of measurement tools are heart rate tools, peak velocity tools, and a number of other tools described below. When the ultrasound system user desires to make a particular measurement the user interface 99 is used to select the measurement tool for that measurement. A typical user interface 60, taken from a touchpanel display of a constructed implementation of the present invention, is shown in FIG. 3. For instance if the user desires to make a heart rate measurement, the user touches the heart rate button 62 on the touchscreen display 60. This selection loads the heart rate tool from the measurement tool store 52 into the measurement processor 50 where the tool is operated to make a heart rate measurement on the Doppler data provided by the waveform peak tracer 42.

The user interface 99 also is used to enter control signals for the measurement processor. Such control signals may include commands such as the selection of a particular cardiac cycle or group of cardiac cycles on which to make a measurement as explained more fully below.

The measurement processor 50 operates on Doppler data to make the measurement desired by the user. The results of the measurement are coupled to a graphics processor 44 from which graphical measurement results are processed for display on and/or with the spectral Doppler data by the display processor 46. As illustrated below, these results may be displayed numerically, graphically, or both.

An automated measurement made in accordance with the principles of the present invention is shown in FIG. 4. In this first example the peak velocities of spectral lines 70 of a spectral display have been traced by the line 80, which identifies the peak velocity of the waveform of each heart cycle. The Doppler waveform can comprise a sequence of dozens or hundreds of heartbeats. This tracing can be done at the time the spectral data is acquired and stored in the Cineloop memory or the tracing can be done at the time the spectral data is to be analyzed. In this example the tracing 80 is visually displayed on the spectral waveform display but it may alternatively be hidden from display if desired. From all of the identified velocity peaks the maximum velocity is chosen as the initial heart cycle on which a measurement is to be made, as clinicians usually begin measurements with the peak velocity cardiac cycle. The cardiac cycle containing this maximum velocity value is highlighted by delineating the beginning and the end of the heart cycle with “goalposts” 92 and 94. In this example the goalposts are placed at successive end diastole points in the cardiac sequence. Since the tool used in this example is a heart rate tool, the tool measures the interval between the goalposts and from this time interval computes the heart rate. This result is shown numerically in the example of FIG. 4 as a heart rate of 84 beats per minute. Thus, in response to the selection of only a spectral data sequence and a specific measurement, in this case the heart rate measurement, the ultrasound system automatically identifies the cardiac cycle with the highest peak velocity and makes the measurement (the heart rate) for this heart cycle. A clinically viable measurement is thus obtained quickly and without the need to scan through the sequence of spectral data or place markers on the data, both time consuming and dexterously taxing exercises.

FIG. 5a illustrates the heart rate measurement being made on a typical ultrasound system display 34. At the top of the display is a B mode image 110 of anatomy containing a blood vessel 114. A cursor line is manipulated over the B mode image until a sample volume cursor 112 on the line is located at the point where spectral Doppler data is to be acquired, in this case in the center of the blood vessel 114. Doppler data is then acquired from this location and displayed as a scrolling spectral display 120 as it is acquired. In this example all of this information has been stored in Cineloop memory and is being analyzed. The first measurement made is the heart rate, which is done for the cardiac cycle containing the maximum peak velocity identified as described above. A portion of the spectral display 120 containing this cardiac cycle is displayed on the screen 34 in response to activation of the heart rate tool by button 62, the goalposts 92 and 94 are placed at the beginning and end of the identified peak velocity cardiac cycle, and the computed heart rate value of 72 bpm is displayed on the screen 34, in this example just to the right of the B mode image 110.

The exemplary user interface of FIG. 3 is seen to contain a button 66 which is marked “Prev/Next Cycle.” This button is used to move the selected cardiac cycle of the spectral display forward or backward on the display, thereby causing a measurement to be made on an adjacent heart cycle to the one currently highlighted on the spectral display 120. If, for example, the right side of the button 66 is touched to move the selected cardiac cycle of FIG. 5a forward to the next heart cycle, the display would appear as shown in FIG. 5b. This illustration shows that the next cardiac cycle is highlighted by the goalpost lines 92 and 94, and that the heart rate for this heart cycle is now displayed, in this example as 70 bpm.

Similarly, if the left side of the button 66 is touched to move the selected cardiac cycle of FIG. 5a to the previous cardiac cycle, the display would appear as shown in FIG. 5c with the previous cardiac cycle highlighted by the goalpost lines 92 and 94 and measured. The Prev/Next Cycle button can be used in conjunction with any measurement of the present invention.

Another example of the present invention is shown in FIGS. 6a-6c for a peak velocity tool. In FIG. 6a the user has selected a peak velocity tool which is designed to identify the peak velocity of a heart cycle. The measurement processor identifies the cardiac cycle with the highest peak velocity value, displays a portion of the Doppler sequence 120 containing that cycle, and places a marker 96 at that peak in the spectral display. In this example the user has opted not to display the goalpost lines. As in the preceding examples, the Prev/Next Cycle button 66 can be actuated to move the selected cardiac cycle forward by one cycle (or more by repetitive actuations) as shown in FIG. 6b, or back a cycle at a time as shown in FIG. 6c.

Another measurement which can be made in accordance with the present invention is a time/slope measurement. A time/slope measurement is made by actuating button 68 on the user interface of FIG. 3, launching the time/slope tool. The result of an acceleration time/slope measurement is shown in FIG. 7. The measurement processor identifies the peak velocity cardiac cycle of the spectral Doppler sequence and places a marker 97 at the end diastole point of the immediately preceding cardiac cycle. A marker 98 is placed at the peak systolic velocity point of the identified heart cycle. In this example a dotted line is displayed between these two points. The measurement processor calculates and displays time and slope values for the interval between the markers 97 and 98, which in this example are a time interval of 79 msec and a slope (rate of change) of 699 cm/sec. Another time/slope measurement which can be made is a deceleration measurement as illustrated in FIG. 8. After placing the peak velocity marker 98 the measurement processor places the second marker 99 at the end systole point of the cardiac waveform which in this example is on a vertical line 199. A dotted line is displayed between the two markers and the time and slope values are calculated and displayed for the marked systolic interval.

Tools can be used to make tracings of the identified peak velocity waveform as shown in FIGS. 9 and 10. A continuous trace 130 is displayed as a series of dots in the example shown in FIG. 9. This trace is essentially the series of points identified on each spectral line by the waveform peak tracer 42 as discussed above. The trace 130 in this example is displayed between end diastole point 97 of the previous heart cycle and the end diastole point 91 of the current cardiac cycle. Another type of tracing which can be made automatically is a trace by points trace 140 as shown in FIG. 10. This tracing is made by connecting key points in the cardiac cycle with straight lines, such as end diastole, peak systole, end systole, mean diastole, and so forth.

Another measurement which can be made in accordance with the present invention is the average heart rate over multiple heart cycles as shown in FIGS. 11 and 12. In the example FIG. 11 the heart rate is calculated by the measurement processor from the interval of the heart cycle between goalpost lines 92 and 94, and the preceding heart cycle bounded by goalpost lines 194 and 92. The numerical result of this two-cycle calculation is shown on the display screen 34. In the example of FIG. 12 four cardiac cycles are used in the heart rate calculation. As the drawing illustrates the four heart cycles used in the calculation are bounded by goalpost lines 194, 92, 94, 192, and 196. Other numbers of cardiac cycles, either sequential or nonsequential, can also be used for these measurements.

Variations of the examples described above are within the scope of the present invention. For example, the user can be given the option to manually adjust the peak velocity tracing or values on which the measurements are to be made, as described in our pending international patent application number IB2005/052572. Another variation is for the waveform peak tracer to identify the peak velocities of the analyzed heart sequence ranging from the highest peak velocity to the lowest peak velocity. A control can be provided for the user to skip from one heart cycle to another in the sequence of the peak velocities. This will enable the user to first view and measure the cardiac cycle with the maximum peak velocity, then the cardiac cycle with the second highest peak velocity, then the cardiac cycle with third highest peak velocity, and so forth. Another variation is to jump directly to the cardiac cycle with the lowest peak velocity. Other variations will readily occur to those skilled in the art.

What is claimed is:

1. An ultrasonic diagnostic imaging system for analyzing blood flow comprising:
means for acquiring spectral Doppler information for a sequence of cardiac cycles;
a spectral Doppler analyzer, responsive to the spectral Doppler information, which acts to automatically identify a cardiac cycle exhibiting a specified characteristic;
a measurement tool, responsive to the spectral Doppler analyzer, which acts to perform a predetermined analysis on the identified cardiac cycle and produce a result;
a user control operable to actuate the measurement tool; and
a display responsive to the measurement tool for displaying the measurement result.

2. The ultrasonic diagnostic imaging system of claim 1, wherein the specified characteristic comprises the maximum peak velocity.

3. The ultrasonic diagnostic imaging system of claim 2, wherein the spectral Doppler analyzer comprises a peak velocity analyzer.

4. The ultrasonic diagnostic imaging system of claim 3, wherein the spectral Doppler analyzer further acts to identify the peak velocity value on the spectral lines of a plurality of heart cycles.

5. The ultrasonic diagnostic imaging system of claim 4, wherein the spectral Doppler analyzer further acts to identify the peak velocity value of each cardiac cycle of a sequence of cardiac cycles.

6. The ultrasonic diagnostic imaging system of claim 5, wherein the spectral Doppler analyzer further acts to identify the maximum velocity value of the peak velocity values of a sequence of cardiac cycles.

7. The ultrasonic diagnostic imaging system of claim 5, wherein the predetermined analysis performs at least one of the following measurements: acceleration/deceleration time or slope, peak velocity, heart rate, average heart rate, Doppler waveform trace, or point to point waveform trace.

8. The ultrasonic diagnostic imaging system of claim 6, wherein the spectral Doppler information for a sequence of cardiac cycles further comprises a scrolling display, only a portion of which can be displayed on the display at a given time; and
wherein the spectral Doppler analyzer automatically causes a portion of the scrolling display to be displayed which includes the maximum velocity value.

9. The ultrasonic diagnostic imaging system of claim 6, wherein the spectral Doppler information for a sequence of cardiac cycles further comprises a scrolling display, only a portion of which can be displayed on the display at a given time; and
wherein the actuation of the measurement tool automatically causes a portion of the scrolling display to be displayed which includes the maximum velocity value.

10. The ultrasonic diagnostic imaging system of claim 1, wherein the measurement result is numerically displayed.

11. The ultrasonic diagnostic imaging system of claim 1, wherein the spectral Doppler analyzer comprises a peak velocity tracer.

12. A method of making a measurement on the ultrasonic spectral Doppler information of a sequence of cardiac cycles comprising:
selecting the ultrasonic spectral Doppler information of a sequence of cardiac cycles;
selecting a measurement tool which acts to make a measurement on the spectral Doppler information;
automatically identifying the cardiac cycle with the maximum peak velocity value; and
making the measurement on the identified cardiac cycle.

13. The method of claim 12, further comprising displaying the result of the measurement.

14. The method of claim 12, wherein selecting a measurement tool is done by a user and causes the step of automatically identifying the cardiac cycle with the maximum peak velocity value to be immediately executed.

15. The method of claim 12, further comprising automatically displaying the cardiac cycle with the maximum peak velocity value following the step of automatically identifying.

16. The method of claim 12, wherein making the measurement further comprises making the measurement on the identified cardiac cycle and at least one adjacent cardiac cycle.

17. The method of claim 12, further comprising manually selecting a cardiac cycle adjacent to the identified cardiac cycle with a user input.

18. A method of making a measurement on the ultrasonic spectral Doppler information of a sequence of cardiac cycles comprising:
selecting the ultrasonic spectral Doppler information of a sequence of cardiac cycles;
selecting a measurement tool which acts to make a measurement on the spectral Doppler information;
automatically identifying the cardiac cycle exhibiting a predetermined characteristic; and
making the measurement on the identified cardiac cycle.

19. The method of claim 18, wherein automatically identifying further comprises automatically identifying the cardiac cycle exhibiting a predetermined velocity characteristic.

20. The method of claim 18, wherein selecting a measurement tool further comprises selecting a measurement tool which performs one of the measurements of acceleration/deceleration time or slope, peak velocity, heart rate, average heart rate, Doppler waveform trace, or point to point waveform trace.
